(19) United States
(12) Patent Application Publication
(10) Pub. No.: US 2023/0089536 A1
(43) Pub. Date: Mar. 23, 2023

(54) COMBINATION OF AN LSD-1 INHIBITOR AND NIVOLUMAB FOR USE IN TREATING SCLC OR SQNSCLC

(71) Applicant: Celgene Quanticel Research, Inc., Summit, NJ (US)

(72) Inventors: Ellen Filvaroff, Summit, NJ (US); Ida Aronchik, Sumit, NJ (US); Tracy Chow, Sumit, NJ (US); Eric Olson, Summit, NJ (US); Brian Fox, Summit, NJ (US); Maria Wang, Summit, NJ (US)

(73) Assignee: Celgene Quanticel Research, Inc., Summit, NJ (US)

(21) Appl. No.: 17/909,292

(22) PCT Filed: Mar. 5, 2021
(86) PCT No.: PCT/US2021/021101

§ 371 Date: Sep. 2, 2022

Related U.S. Application Data
(60) Provisional application No. 62/986,541, filed on Mar. 6, 2020.

(51) Int. Cl. A61K 31/496; A61K 9/48; A61K 9/20; A61K 9/00; A61P 35/00

(52) U.S. Cl. CPC A61K 31/496; A61K 9/48; A61K 9/20; A61K 9/0019; A61P 35/00

(57) ABSTRACT

The present application relates generally to a lysine specific demethylase-1 (LSD-1) inhibitor, or a pharmaceutically acceptable salt thereof, and nivolumab, for use in methods for treating small cell lung cancer (SCLC) and/or squamous non-small cell lung cancer (sqNSCLC).

### RELATED APPLICATIONS

This application claims the priority benefit of U.S. provisional application 62/986,541 filed Mar. 6, 2020, the entire contents of which are incorporated herein by reference.

### FIELD

The present application relates generally to methods for treating small cell lung cancer (SCLC) and/or squamous non-small cell lung cancer (sqNSCLC) with a combination of a lysine specific demethylase-1 (LSD-1) inhibitor, or a pharmaceutically acceptable salt thereof, and nivolumab.

### BACKGROUND

Lung cancer is the most common cancer worldwide with approximately 1.8 million new diagnoses and 1.59 million deaths in 2012, which corresponds to the third highest incidence among cancers and the most common cancer-related mortality.

Small cell lung cancer is an aggressive high-grade neuroendocrine tumor associated with a short doubling time, a high growth fraction, and early development of widespread metastases, which contribute to the extremely poor disease prognosis. The World Health Organization (WHO) divides lung cancer into 2 major classes based on its biology, therapy, and prognosis: small cell lung cancer (SCLC) and non-small cell lung cancer (NSCLC).

Small cell lung cancer is an aggressive high-grade neuroendocrine tumor associated with a short doubling time, a high growth fraction, and early development of widespread metastases, which contribute to the extremely poor disease prognosis. These aspects of SCLC, as well as the limited success of current treatments, highlight the unmet medical need for the development of new therapeutics for SCLC, particularly in relapsed disease.

Non-small cell lung cancer accounts for 80% to 90% of lung cancers and includes two major types: (1) non-squamous carcinoma (including adenocarcinoma, large-cell carcinoma, other cell types); and (2) squamous cell (epidermoid) carcinoma. Squamous histology is associated with shorter survival than non-squamous histology.

Advanced squamous non-small cell lung cancer (sqNSCLC) remains a recalcitrant disease. While non-squamous NSCLC has benefited from advances in chemotherapy doublets (pemetrexed and platinum), VEGF targeted therapy (bevacizumab) and tumor profiling with actionable mutations for therapeutic interventions (ie, EGFRmut, ALK, BRAF, ROS1), the same has not occurred in the setting of sqNSCLC. Together, these factors make sqNSCLC an especially challenging disease to such that new therapies, especially ICI and combinations, could have a large impact.

Lung cancer can be asymptomatic at early stages. As such, most patients are diagnosed at an advanced stage that is not curable by surgery and have poor prognoses. Despite recent advances in targeted and immune mediated therapy, such as anti-programmed cell death 1 (PD1)/programmed death-ligand 1 (PD-L1), most patients with solid tumors do not achieve long term disease control. While cytotoxic chemotherapy remains an important disease control modality in both first and second-line treatment for patients with SCLC, long term disease control is limited. Further, while chemotherapy, targeted therapy, and/or anti-PD-1/PD-L1 therapies provide long term benefit to NSCLC patients, the majority of NSCLC patients will ultimately progress due to resistance mechanisms and succumb due to the disease.

Thus, there remains a need for more effective treatments for small cell lung cancer (SCLC) and/or squamous non-small cell lung cancer (sqNSCLC), and this disclosure satisfies this need.

### SUMMARY

The present application relates generally to methods for treating small cell lung cancer (SCLC) and/or squamous non-small cell lung cancer (sqNSCLC). The methods comprise administering a combination of a lysine specific demethylase-1 (LSD-1) inhibitor, or a pharmaceutically acceptable salt thereof, with nivolumab.

The aspects and embodiments of the present disclosure provide for methods for treating subjects with small cell lung cancer (SCLC) and/or squamous non-small cell lung cancer (sqNSCLC).

Provided in one aspect is a method of treating a subject having small cell lung cancer (SCLC) and/or squamous non-small cell lung cancer (sqNSCLC) comprising: (a) administering to the subject an LSD-1 inhibitor; and (b) concomitantly or sequentially administering nivolumab; wherein the LSD-1 inhibitor is a compound having the structure:

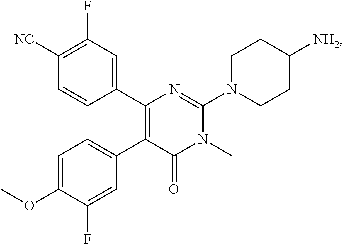 [SMILES: COc1ccc(-c2c(-c3ccc(C#N)c(F)c3)nc(N3CCC(N)CC3)n(C)c2=O)cc1F]

or a besylate salt thereof. In some embodiments the nivolumab is administered before the LSD-1 inhibitor, in some embodiments the nivolumab is administered after the LSD-1 inhibitor, and in some embodiments the nivolumab is administered simultaneously with the LSD-1 inhibitor.

In some embodiments, the subject has any one of the following: (a) a complete response (CR) as assessed by Response Evaluation Criteria in Solid Tumor (RECIST), Version 1.1; (b) the disappearance of all target lesions; and/or (c) the reduction of target and/or non-target pathological lymph nodes in short axis to less than about 10 mm.

In some embodiments, the subject has any one of the following: (a) a partial response (PR) as assessed by Response Evaluation Criteria In Solid Tumor (RECIST), Version 1.1; and/or (b) at least a 30% decrease in the sum of diameters of target lesions, taking as reference the baseline sum diameter.

In some embodiments, the subject has a duration of response as defined by a time from the first occurrence of a documented objective response to a time of a first objectively documented progression, as determined by Response Evaluation Criteria In Solid Tumor (RECIST), Version 1.1, or death from any cause, whichever comes first, wherein the duration of the response is: (a) about 1, about 2, about 5, about 10, about 52, or greater weeks; (b) at least about 1 week, at least about 2 weeks, at least about 3 weeks, at least about 4 weeks, at least about 5 weeks, at least about 6 weeks, at least about 12 weeks, at least about 18 weeks, at least about 24 weeks, at least about 30 weeks, at least about 36 weeks, at least about 42 weeks, at least about 48 weeks, or at least about 54 weeks; and/or (c) about 1 week, about 2 weeks, about 3 weeks, about 4 weeks, about 5 weeks, about 6 weeks, about 12 weeks, about 18 weeks, about 24 weeks, about 30 weeks, about 36 weeks, about 42 weeks, about 48 weeks, or about 54 weeks.

In some embodiments, the subject has a progression-free survival as defined by from first dose of study treatment to the date of the first objectively documented tumor progression as determined by Response Evaluation Criteria In Solid Tumor (RECIST), Version 1.1, or death from any cause, whichever comes first, wherein the duration of the progression-free survival is: (a) about 1, about 2, about 5, about 10, about 52, or greater weeks; (b) at least about 1 week, at least about 2 weeks, at least about 3 weeks, at least about 4 weeks, at least about 5 weeks, at least about 6 weeks, at least about 12 weeks, at least about 18 weeks, at least about 24 weeks, at least about 30 weeks, at least about 36 weeks, at least about 42 weeks, at least about 48 weeks, or at least about 54 weeks; and/or (c) about 1 week, about 2 weeks, about 3 weeks, about 4 weeks, about 5 weeks, about 6 weeks, about 12 weeks, about 18 weeks, about 24 weeks, about 30 weeks, about 36 weeks, about 42 weeks, about 48 weeks, or about 54 weeks.

In some embodiments, the method further comprises any one of the following: (a) the LSD-1 inhibitor is administered orally; (b) the LSD-1 inhibitor is administered in the form of a tablet or capsule; (c) the LSD-1 inhibitor is administered once a week; and/or (d) the LSD-1 inhibitor is administered at a dose of about 20 mg, about 40 mg, or about 60 mg.

In some embodiments, (a) the LSD-1 inhibitor is administered at about 40 mg orally once a week in a 28-day period; and/or (b) the LSD-1 inhibitor is administered on Days 1, 8, 15, and 22 in a 28-day period; and/or (c) the 28 day period is repeated for as long as the subject has a complete response (CR) or partial response (PR) as assessed by Response Evaluation Criteria In Solid Tumor (RECIST), Version 1.1.

In some embodiments, (a) the LSD-1 inhibitor is administered at about 60 mg orally once a week in a 28-day period; and/or (b) the LSD-1 inhibitor is administered on Days 1, 8, 15, and 22 in a 28-day period; and/or (c) the 28 day period is repeated for as long as the subject has a complete response (CR) or partial response (PR) as assessed by Response Evaluation Criteria In Solid Tumor (RECIST), Version 1.1.

In some embodiments, (a) the nivolumab is administered intravenously; and/or (b) the nivolumab is administered in the form of an injection; and/or (c) the nivolumab is administered once every two weeks or every 4 weeks; and/or (d) the nivolumab is administered at a dose of at least about 240 mg or about 480 mg; and/or (e) the nivolumab is administered at a dose of about 240 mg or about 480 mg.

In some embodiments, (a) the nivolumab is administered at about 480 mg intravenously once a week in a 28-day period; and/or (b) the nivolumab is administered on Day 1 in a 28-day period; and/or (c) the 28 day period is repeated for as long as the subject has a complete response (CR) or partial response (PR) as assessed by Response Evaluation Criteria In Solid Tumor (RECIST), Version 1.1.

In some embodiments, the subject exhibits one or more of the following specific levels of baseline characteristics: (a) gene expression in peripheral blood (LSD1-regulated genes in PBMCs) and/or in tumor samples (such as SOX-2, Notch1/2, ASCL1, POU2F2, YAP, NeuroD1, CgA, GRP, REST, HES1, HEY1); and/or (b) molecular features in tumor samples, such as amplification of Sox-2 (c) secreted proteins in blood selected from pro-gastrin-releasing peptide (pro-GRP) and chromogranin A (CgA) and midkine; and/or (d) localization and/or density of T cells, MDSCs and other immune cells in tumor tissues; and/or (e) expression of programmed cell death protein 1 (PD-1) and/or programmed death-ligand 1 (PD-L1) in tumor tissues; and/or (f) expression of lysine-specific histone demethylase 1A (LSD1) and/or an LSD1-associated molecular signature in tumor tissue; and/or (g) protein markers in tumor tissues and/or in circulating tumor cells (CTCs); and/or (h) amount and molecular features of circulating tumor DNA (ctDNA) in the blood.

In some embodiments, the subject exhibits one or more of the following changes from baseline: (a) gene expression in peripheral blood (LSD1-regulated genes in PBMCs) and/or in tumor samples (such as SOX-2, Notch1, ASCL1, IGFBP2/5, REST, Hes1, Hey1, MDK, CgA, GRP); and/or (b) secreted proteins in blood selected from pro-gastrin-releasing peptide (pro-GRP) and chromogranin A (CgA) and midkine; and/or (c) localization and/or density of T cells, MDSCs and other immune cells in tumor tissues; and/or (d) expression of programmed cell death protein 1 (PD-1) and/or programmed death-ligand 1 (PD-L1) in tumor tissues; and/or (e) expression of lysine-specific histone demethylase 1A (LSD1) and/or an LSD1-associated molecular signature in tumor tissue; and/or (f) protein markers in tumor tissues (such as CXCL9, MCSF, Notch1/2, chromagraninA) and/or in circulating tumor cells (CTCs); and/or (g) amount and molecular features of circulating tumor DNA (ctDNA) in the blood. In some embodiments, the gene expression in tumor samples is the gene expression of sex determining region Y-box 2 (SOX2).

In some embodiments, the protein marker in tumor tissues is one or more of AC124319.1, ADAR, APOL6, ARID5B, ARL4A, ASCL1, AUTS2, B2M, BANK1, BATF2, BPGM, BST2, BTG1, C1R, C1S, CASP1, CASP3, CASP4, CASP7, CASP8, CHGA, CCL2, CCL5, CCL7, CD274 (PDL1), CD3, CD38, CD4, CD40, CD69, CD74, CD8, CD86, CDH2, CDKN1A, CFB, CFH, CIITA, CMKLR1, CMPK2, CMTR1, CSF2RB, CXCL10, CXCL11, CXCL9, DDX58, DDX60, DHX58, DLL1, DLL3, EIF2AK2, EIF4E3, EPSTI1, FAS, FCGR1A, FGFR1, FGFR13, FGL2, FPR1, GBP4, GBP6, GCH1, GPR18, GRP, GZMA, HELZ2, HERC6, HEY1, HES1, HIF1A, HLA-A, HLA-B, HLA-DMA, HLA-DQA1, HLA-DRB1, HLA-G, ICAM1, IDOL IFI27, IFI30, IF135, IF144, IFI44L, IFIH1, IFIT1, IFIT2, IFIT3, IFITM2, IFITM3, IFNAR2, IFL10RA, IGFBP2, IGFBP5, IL15, IL15RA, IL18BP, IL2RB, IL4R, IL6, IL7, IRF1, IRF2, IRF4, IRF5, IRF7, IRF8, IRF9, ISG15, ISG20, ISOC1, ITGAB, ITGB7, JAG1, JAK2, KAT2B, KLRK1, LAP3, LATS2, LCP2, LGALS3BP, LYSE, LYSMD2, MAGEC2, 1-MAR, MCSF, MDK, METTL7B, MT2A, MEHFD2, MVP, MX1, MX2, MYD88, M-CSF, NAMPT, NCOA3, NEUROD1, NFKB1, NFKBIA, NLRC5, NMI, NOD1, NOTCH1, NOTCH2, NUP93, OAS2, OAS3, OASL, OGFR, P2RY14, PARP12, PARP14, PDE4B, PD1, PELI1, PFKP, PIM1, PLA2G4A, PLSCR1, PML, PNP, PNPT1, POU2F3, PSMA2, PSMA3, PSMB10, PSMB2, PSMB8, PSMB9, PSME1, PSME2, PTGS2, PTPN1, PTPN2, PTPN6, RAPGEF6, RBCK1, RCOR2, REST, RIPK1, RIPK2, RNF31, RSAD2, RTP4, SAMD9L, SAMHD1, SECTM1, SELP, SERPINGLSGK1, SLAMF7, SLC25A28, SOCS1, SOCS3, SOD2, SOX2, SP110, SPPL2A, SRI, SSPN, ST3GAL5, ST8SIA4, STAT1, STAT2, STAT3, STAT4, TAP1, TAPBP, TDRD7, THBS1, TNFAIP2, TNFAIP3, TNFAIP6, TNFAIP10, TOR1B, TRAFD1, TRIM14, TRIM 21, TRIM25, TRIM 26, TXNIP, UBE2L6, UPP1, USP18, VAMP5, VAMP8, VEGF, VCAM1, WARS, WNT11, XAF1, XCL1, ZBP1, ZEB1, ZEB2, or ZNFX1.

Both the foregoing summary and the following description of the drawings and detailed description are exemplary and explanatory. They are intended to provide further details of the invention, but are not to be construed as limiting. Other objects, advantages, and novel features will be readily apparent to those skilled in the art from the following detailed description of the invention.

### DETAILED DESCRIPTION

### I. Overview

The present invention is directed to treating small cell lung cancer (SCLC) and/or squamous non-small cell lung cancer (sqNSCLC) by administering a combination of a lysine specific demethylase-1 (LSD-1) inhibitor, or a pharmaceutically acceptable salt thereof, with a PD-1 inhibitor, such as nivolumab.

In some embodiments the nivolumab is administered before the LSD-1 inhibitor, in some embodiments the nivolumab is administered after the LSD-1 inhibitor, and in some embodiments the nivolumab is administered simultaneously with the LSD-1 inhibitor.

Immune checkpoint inhibition has changed the treatment paradigm for many cancer types by unleashing a patient's own immune systems against tumors. Immune checkpoint inhibitors (ICIs) are a class of immunotherapeutic agents that restore the exhausted host's antitumor immune responses mediated by the tumors. In other words, the effectiveness of ICIs is grounded on a pre-existent anti-tumoral cellular immune response, which is usually recognized by the presence of tumor T-lymphocytic infiltrates that are, however, most often ineffective because of expression of co-inhibitory (or checkpoint) receptors such as PD-1, CTLA-4, and others. Blocking these checkpoint receptors or their ligands restores T cell function and leads to clinical responses.

One of the most widely investigated immune-checkpoint therapy pathways is the programmed cell death protein 1 (PD-1)/programmed cell death protein ligand 1 (PD-L1) pathway. PD-1 is expressed on activated CD8+ T cells, as well as B cells and natural killer cells, in the setting of chronic antigen exposure. PD-1 ligand (PD-L1) expression is induced by localized inflammatory stimuli, such as interferons released by the infiltrating T cells. Although PD-1 and PD-L1 checkpoint blockade can result in dramatic therapeutic responses, this therapy is effective only in a subset of subjects, and many of them are only partial responders to therapy. Subjects who do not respond to initial therapy with PD-1/PD-L1 blockade are referred to as having “primary resistance” to therapy. Furthermore, a growing subset of subjects show robust initial response to therapy, but later have progressive disease. This phenomenon, in which the disease is either refractory to resumption of therapy or develops despite continuation of therapy, is known as “acquired resistance” to PD-1/PD-L1 blockade immunotherapy. In all, almost four-fifths of patients either do not respond to or lose their responsiveness to ICI.

With regard to lung cancer, lung cancer cells overexpress PD-L1 as a mechanism for suppressing T-cell response, immune checkpoint blockade with PD-1/programmed cell death ligand 1 (PD-L1) inhibitors has become part of the standard-of-care treatment option for patients with advanced stage NSCLC; however, only a small subset (20-30%) of patients respond to treatment (Jain et al., Ther. Adv. Respir. Dis., 12:1-13) (2018)). For SCLC, checkpoint inhibitors have demonstrated some efficacy, but the magnitude of benefit has been relatively modest, and only a subset of patients respond to treatment.

In other words, the use of immune checkpoint inhibitors (ICIs) in the treatment of lung cancer has proven efficacy as demonstrated by the increasing overall survival (OS), progression-free survival (PFS), overall response rate (ORR) and longer duration of response (DoR) as compared to chemotherapy alone. However, only a limited number of subjects have long term benefit with ICI treatment. These limitations have made it necessary to explore combination treatment methods which are generally aimed at enhancing or activating antitumor immunity.

The tumor microenvironment can encompass multiple immunosuppressive mechanisms, including dysfunctional T cells and lack of T cell infiltration or recognition by T cells, which prevents subjects from responding to anti-PD-1/PD-L1 therapy. These mechanisms provide a basis for selecting appropriate combinations to complement the anti-PD-1/PD-L1 action. For example, the presence of T cytotoxic tumor infiltrates (defining the so-called “hot tumors”) justifies targeting other checkpoint inhibitors and enhancing anti-tumor immune response. With modest and immunosuppressed infiltrates, new therapy could be aimed at inhibitory mediators (such as TGF-β, IL-10, etc), immune suppressive cells (such as myeloid derived suppressor cells, regulatory lymphocyte T cells), or immune ignored cancer stem cells. Also, when T cells are excluded from the tumor bed and accumulate at the tumor border, then potentially effective combinations might be aimed at reactivating or supplanting T cell recruiting signals (eg, chemokines). Finally, when T cells are absent (“cold tumors”), then various modalities to increase tumor immunogenicity and restart antigen-presentation or T cell priming might prove useful.

This disclosure recognizes that LSD-1 inhibitors, such as CC-90011 disclosed herein, are compatible for use in a combination treatment with an immune checkpoint inhibitor for treating lung cancer. This is because: (1) inhibition of LSD1 reduces cell proliferation and stem cell maintenance while promoting cell differentiation and reducing tumor growth in preclinical models; and (2) LSD-1 inhibitors may have potential immunomodulatory effects via their abilities to impact lymphocytic infiltrates and immunogenicity of tumors. In addition, tumors which lack T cell infiltration (cold tumors) have higher mRNA expression of LSD1 and/or an associated signature, and this may be used to identify patients susceptible to the action of an LSD-1 inhibitor with ICI.

Although checkpoint inhibitors have demonstrated some efficacy in SCLC, the magnitude of benefit has been relatively modest, and only a subset of patients respond. Explanation of this resistance may be attributed to the nature the SCLC tumor with characteristics of a “cold tumor:” limited PD-L1 expression, decreased major histocompatibility complex 1 (MHC1) expression, activation or accumulation of suppressors cells, myeloid derived suppressor cells, regulatory lymphocytes, ineffective priming or activation of dendritic cells and T lymphocytes, and finally low rate of immune cell infiltration. As explained above, using LSD-1 inhibitors may be able to modify one of these characteristics and have the potential to enhance ICI activity. For instance, the low abundance of T cells in SCLC tumors may be one of the reasons why checkpoint inhibitors have limited efficacy. An LSD-1 inhibitor may reverse this phenotype by allowing T cells to infiltrate the tumor. Preclinical data in the literature and generated by the inventors of this disclosure, including survival data in mice with tumors, suggest that an LSD-1 inhibitor could induce pro-inflammatory and T cell permissive changes in the tumor microenvironment and, thus, enhance efficacy of checkpoint inhibitors such as nivolumab.

This disclosure recognizes that LSD-1 inhibition sequentially or in combination with cytoreductive therapy improves disease-free survival by preventing the emergence of resistant clones through treatment of tumorigenic stem cells. This mechanism of action is applicable to many earlier stages of solid tumors where existing standard of care does not result in long-term disease control for all patients. In addition, the LSD-1 compound has a role in treating tumors resistant to current immune check-point blockade, an area of high unmet need for multiple solid tumors where ICIs are either not effective, or where ICIs are currently employed.

This disclosure recognizes that in some embodiments, an LSD-1 mitigates primary and acquired resistance to ICI, due to its expected reversal of the cancer stem cell (CSC) phenotype and T and immune cell exclusion in SCLC and sqNSCLC.

In this disclosure, “ICI” refers to both anti-PD-1 or anti-PD-L1 treatments. As evidenced by the results from Example 2 of this disclosure, in some embodiments, an LSD-1 inhibitor increases the response rates in 3 different lung cancer populations: (i) PD-1 inhibitor naïve (Cohort A, SCLC), (ii) a PD-1 inhibitor “experienced” (Cohort B, SCLC; and (iii) Cohort C, sqNSCLC) when given in combination with nivolumab. In some embodiments, an LSD-1 inhibitor enhances nivolumab responses in “cold tumor” phenotypes (Cohort A, B, and C). In some embodiments, tumors with higher expression of an LSD1-associated molecular signature and low T infiltrating lymphocytes (TILs) would have the best response to the combination of ICI with an LSD-1 inhibitor, based on the LSD-1 inhibitor hypothesized mechanism of action of increasing T cell infiltration into tumors. In some embodiments, the LSD-1 inhibitor mitigates acquired resistance to ICI in SCLC as well as sqNSCLC (Cohorts A, B and C). For Cohorts B and C, the trial enrolls subjects who have an initial response or stable disease to ICI, but progress within the first 9 months after completion of the chemotherapy treatment. In some embodiments, the patients in any of the three lung cancer populations have been previously treated and/or currently receiving chemotherapy (e.g., platinum-based chemotherapy or a platinum-based chemotherapy doublet). In some embodiments, there is a synergistic effect observed with treatment of the LSD-1 inhibitor with nivolumab in any one of the three different lung cancer populations described in Example 2.

### II. LSD-1 Inhibitor

In the embodiments described herein, the LSD-1 inhibitor is a compound having the structure:

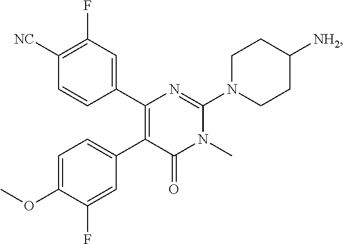 [SMILES: COc1ccc(-c2c(-c3ccc(C#N)c(F)c3)nc(N3CCC(N)CC3)n(C)c2=O)cc1F]

or a pharmaceutically acceptable salt thereof, such as the besylate salt. The chemical name of the above compound is 4-[2-(4-Amino-piperidin-1-yl)-5-(3-fluoro-4-methoxy-phenyl)-6-oxo-1,6-dihydro-pyrimidin-4-yl]-2-fluoro-benzonitrile, with a chemical formula of C₂₃H₂₁F₂N₅O₂, molecular weight of 437.44, and CAS number of 1821307-10-1. 4-[2-(4-amino-piperidin-1-yl)-5-(3-fluoro-4-methoxy-phenyl)-1-methyl-6-oxo-1,6-dihydro-pyrimidin-4-yl]-2-fluoro-benzonitrile is described in U.S. patent application Ser. No. 14/701,304 (U.S. Pat. No. 9,255,097).

In any of the embodiments described herein, the LSD-1 inhibitor can be administered orally. Oral doses can typically range from about 1.0 mg to about 1000 mg, one to four times, or more, per day. In some embodiments, the LSD-1 inhibitor is administered in about 20 mg, about 40 mg, or about 60 mg doses. In any of the embodiments described herein, the LSD-1 inhibitor can be administered in the form of a tablet or capsule. In any of the embodiments described herein, the LSD-1 inhibitor can be administered once a week. In any of the embodiments described herein, the LSD-1 inhibitor can be administered at a dose of about 20 mg. In any of the embodiments described herein, the LSD-1 inhibitor can be administered at a dose of about 40 mg. In any of the embodiments described herein, the LSD-1 inhibitor can be administered at a dose of about 60 mg.

### III. Nivolumab

Antibodies that could interrupt mechanisms by which tumors evade the immune system may be used as immune checkpoint inhibitors. For example, anti-PD-1 antibodies block the interaction of PD-1 with PD-L1 and PDL-2 and can be used as PD-1/programmed cell death ligand 1 (PD-L1) inhibitors.

Suitable anti-PD-1 antibodies include isolated antibodies that bind specifically to human PD-1 and cross-compete for binding to human PD-1 with any anti-PD-1 antibody disclosed herein. In some embodiments, the anti-PD-1 antibody binds the same epitope as any of the anti-PD-1 antibodies described herein. The ability of antibodies to cross-compete for binding to an antigen indicates that these monoclonal antibodies bind to the same epitope region of the antigen and sterically hinder the binding of other cross-competing antibodies to that particular epitope region. These cross-competing antibodies are expected to have functional properties very similar those of the reference antibody, e.g., nivolumab, by virtue of their binding to the same epitope region of PD-1. Cross-competing antibodies can be readily identified based on their ability to cross-compete with nivolumab in standard PD-1 binding assays such as Biacore analysis, ELISA assays or flow cytometry.

In certain embodiments, the antibodies that cross-compete for binding to human PD-1, or bind to the same epitope region of human PD-1 antibody, nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Suitable anti-PD-1 antibodies include antigen-binding portions of the above antibodies. It has been amply demonstrated that the antigen-binding function of an antibody can be performed by fragments of a full-length antibody.

Suitable anti-PD-1 antibodies include antibodies that bind to PD-1 with high specificity and affinity, block the binding of PD-L1 and or PD-L2, and inhibit the immunosuppressive effect of the PD-1 signaling pathway. As used herein, an anti-PD-1 “antibody” includes an antigen-binding portion or fragment that binds to the PD-1 receptor and exhibits the functional properties similar to those of whole antibodies in inhibiting ligand binding and up-regulating the immune system. In certain embodiments, the anti-PD-1 antibody or antigen-binding portion thereof cross-competes with nivolumab for binding to human PD-1.

Examples of suitable anti-PD-1 antibodies include but are not limited to, nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; also known as toripalimab; see Si-Yang Liu et al., J. Hematol. Oncol. 10:136 (2017)), BGB-A317 (Beigene; also known as Tislelizumab; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANB011; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), BCD-100 (Biocad; Kaplon et al., mAbs 10(2):183-203 (2018), and IBI308 (Innovent; see WO 2017/024465, WO 2017/025016, WO 2017/132825, and WO 2017/133540).

In certain embodiments, an anti-PD-L1 antibody is substituted for the anti-PD-1 antibody in any of the methods disclosed herein. Examples of suitable anti-PD-L1 antibodies, include but are not limited to, BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), BGB-A333 (BeiGene; see Desai et al., JCO 36 (15suppl):TPS3113 (2018)), and CK-301 (Checkpoint Therapeutics; see Gorelik et al., AACR:Abstract 4606 (April 2016)).

In one embodiment, the anti-PD-1 antibody is nivolumab. Nivolumab (5C4, BMS-936558, MDX-1106, and ONO-4538) is a fully human IgG4 (S228P) PD-1 immune checkpoint inhibitor antibody that selectively prevents interaction with programmed death receptor-1 (PD-1) ligands (PD-L1 and PD-L2), thereby blocking the down-regulation of antitumor T-cell functions (U.S. Pat. No. 8,008,449; Wang et al., Can. Immunol. Res., 2(9):846-56) (2014)).

Nivolumab is marketed as OPDIVO®, which is an injection that is administered as an intravenous infusion over 30 minutes. Nivolumab is marketed for the treatment of patients with unresectable or metastatic melanoma as a single agent or in combination with ipilimumab; patients with melanoma with lymph node involvement or metastatic disease who have undergone complete resection in the adjuvant setting; patients with metastatic non-small cell lung cancer and progression on or after platinum-based chemotherapy, where patients with EGFR or ALK genomic tumor aberrations should have disease progression on FDA-approved therapy for these aberrations prior to receiving OPDIVO®; patients with metastatic small cell lung cancer with progression after platinum-based chemotherapy and at least one other line of therapy; patients with advanced renal cell carcinoma who have received prior antiangiogenic therapy; patients with intermediate or poor risk previously untreated advanced renal cell carcinoma in combination with ipilimumab; adult patients with classical Hodgkin lymphoma that has relapsed or progressed after (i) autologous hematopoietic stem cell transplantation (HSCT) and brentuximab vedotin, or (ii) 3 or more lines of systemic therapy that includes autologous HSCT; patients with recurrent or metastatic squamous cell carcinoma of the head and neck with disease progression on or after a platinum-based therapy; patients with locally advanced or metastatic urothelial carcinoma who have (i) disease progression during or following platinum-containing chemotherapy, or (ii) have disease progression within 12 months of neoadjuvant or adjuvant treatment with platinum-containing chemotherapy; adult and pediatric (12 years and older) patients with microsatellite instability-high (MSI-H) or mismatch repair deficient (dMMR) metastatic colorectal cancer that has progressed following treatment with a fluoropyrimidine, oxaliplatin, and irinotecan as a single agent or in combination with ipilimumab; and patients with hepatocellular carcinoma who have been previously treated with sorafenib.

For metastatic non-small cell lung cancer, nivolumab is administered intravenously (IV) at 240 mg every 2 weeks or 480 mg every 4 weeks. For small cell lung cancer, nivolumab is administered intravenously (IV) at 240 mg every 2 weeks.

In some embodiments, nivolumab is administered at a flat dose of about 240 mg intravenously (IV) once about every 2 weeks. In some embodiments, nivolumab is administered at a flat dose of about 240 mg intravenously (IV) once about every 3 weeks. In some embodiments, nivolumab is administered at a flat dose of about 360 mg intravenously (IV) once about every 3 weeks. In some embodiments, nivolumab is administered at a flat dose of about 480 mg intravenously (IV) once about every 4 weeks.

### IV. Methods of Treatment

The present application provides a method of treating a subject having small cell lung cancer (SCLC) and/or squamous non-small cell lung cancer (sqNSCLC) comprising: (a) administering to the subject an LSD-1 inhibitor; and (b) concomitantly administering nivolumab; wherein the LSD-1 inhibitor is a compound having the structure:

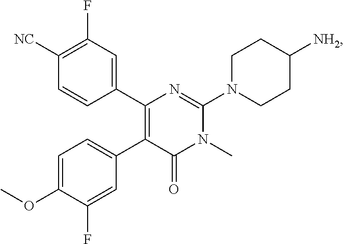 [SMILES: COc1ccc(-c2c(-c3ccc(C#N)c(F)c3)nc(N3CCC(N)CC3)n(C)c2=O)cc1F]

or a besylate salt thereof.

In some embodiments, the subject has a complete response (CR) as assessed by Response Evaluation Criteria In Solid Tumor (RECIST), Version 1.1. In some embodiments, the subject has a complete response (CR) where all target lesions have disappeared. In some embodiments, the subject has a complete response (CR) where target and/or non-target pathological lymph nodes in short axis are reduced to less than about 10 mm.

In some embodiments, the subject has a partial response (PR) as assessed by Response Evaluation Criteria In Solid Tumor (RECIST), Version 1.1. In some embodiments, the subject has a partial response (PR), where there is at least a 30% decrease in the sum of diameters of target lesions, taking as reference the baseline sum diameter.

In some embodiments, the subject has a duration of response as defined by a time from the first occurrence of a documented objective response to a time of a first objectively documented progression, as determined by Response Evaluation Criteria In Solid Tumor (RECIST), Version 1.1, or death from any cause, whichever comes first. In some embodiments, the duration of the response is about 1, about 2, about 5, about 10, about 52, or greater weeks. In some embodiments, the duration of the response is at least about 1 week, at least about 2 weeks, at least about 3 weeks, at least about 4 weeks, at least about 5 weeks, at least about 6 weeks, at least about 12 weeks, at least about 18 weeks, at least about 24 weeks, at least about 30 weeks, at least about 36 weeks, at least about 42 weeks, at least about 48 weeks, or at least about 54 weeks. In some embodiments, the duration of the response is about 1 week, about 2 weeks, about 3 weeks, about 4 weeks, about 5 weeks, about 6 weeks, about 12 weeks, about 18 weeks, about 24 weeks, about 30 weeks, about 36 weeks, about 42 weeks, about 48 weeks, or about 54 weeks.

In some embodiments, the subject has a progression-free survival as defined by from first dose of study treatment to the date of the first objectively documented tumor progression as determined by Response Evaluation Criteria In Solid Tumor (RECIST), Version 1.1, or death from any cause, whichever comes first. In some embodiments, the duration of the progression-free survival is about 1, about 2, about 5, about 10, about 52, or greater weeks. In some embodiments, the duration of the progression-free survival is at least about 1 week, at least about 2 weeks, at least about 3 weeks, at least about 4 weeks, at least about 5 weeks, at least about 6 weeks, at least about 12 weeks, at least about 18 weeks, at least about 24 weeks, at least about 30 weeks, at least about 36 weeks, at least about 42 weeks, at least about 48 weeks, or at least about 54 weeks. In some embodiments, the duration of the progression-free survival is about 1 week, about 2 weeks, about 3 weeks, about 4 weeks, about 5 weeks, about 6 weeks, about 12 weeks, about 18 weeks, about 24 weeks, about 30 weeks, about 36 weeks, about 42 weeks, about 48 weeks, or about 54 weeks.

In some embodiments, the LSD-1 inhibitor is administered orally. In some embodiments, the LSD-1 inhibitor is administered in the form of a tablet or capsule. In some embodiments, the LSD-1 inhibitor is administered once a week. In some embodiments, the LSD-1 inhibitor is administered at a dose of about 20 mg, about 40 mg, or about 60 mg. In some embodiments, the LSD-1 inhibitor is administered at a dose of about 20 mg. In some embodiments, the LSD-1 inhibitor is administered at a dose of about 40 mg. In some embodiments, the LSD-1 inhibitor is administered at a dose of about 60 mg.

In some embodiments, the LSD-1 inhibitor is administered at about 20 mg, 40 mg, or 60 mg orally once a week in a 28-day period. In some embodiments, the LSD-1 inhibitor is administered at about 20 mg orally once a week in a 28-day period. In some embodiments, the LSD-1 inhibitor is administered at about 40 mg orally once a week in a 28-day period. In some embodiments, the LSD-1 inhibitor is administered at about 60 mg orally once a week in a 28-day period. In some embodiments, the LSD-1 inhibitor is administered on Days 1, 8, 15, and 22 in a 28-day period. In some embodiments, the 28 day period is repeated for as long as the subject has a complete response (CR) or partial response (PR) as assessed by Response Evaluation Criteria In Solid Tumor (RECIST), Version 1.1.

In some embodiments, the nivolumab is administered intravenously (IV). In some embodiments, the nivolumab is administered in the form of an injection. In some embodiments, the nivolumab is administered once every two weeks or every 4 weeks. In some embodiments, the nivolumab is administered at a dose of at least about 240 mg or about 480 mg intravenously (IV). In some embodiments, the nivolumab is administered at a dose of at least about 240 mg intravenously (IV). In some embodiments, the nivolumab is administered at a dose of at least about 480 mg intravenously (IV).

In some embodiments, the nivolumab is administered at about 240 mg intravenously once a week in a 28-day period (also referred herein as a “cycle”). In some embodiments, the nivolumab is administered at about 480 mg intravenously once a week in a 28-day period (also referred herein as a “cycle”). In some embodiments, the nivolumab is administered on Day 1 in a 28-day period. In some embodiments, the 28-day period is repeated for as long as the subject has a complete response (CR) or partial response (PR) as assessed by Response Evaluation Criteria In Solid Tumor (RECIST), Version 1.1.

In some embodiments, the subject exhibits one or more of the following specific levels of baseline characteristics: (a) gene expression in peripheral blood (LSD1-regulated genes in PBMCs) and/or in tumor samples (such as SOX-2, Notch1/2, ASCL1, POU2F2, YAP, NeuroD1, CgA, GRP, REST, HES1, HEY1); and/or (b) molecular features in tumor samples, such as amplification of Sox-2 (c) secreted proteins in blood selected from pro-gastrin-releasing peptide (pro-GRP) and chromogranin A (CgA) and midkine; and/or (d) localization and/or density of T cells, MDSCs and other immune cells in tumor tissues; and/or (e) expression of programmed cell death protein 1 (PD-1) and/or programmed death-ligand 1 (PD-L1) in tumor tissues; and/or (f) expression of lysine-specific histone demethylase 1A (LSD1) and/or an LSD1-associated molecular signature in tumor tissue; and/or (g) protein markers in tumor tissues and/or in circulating tumor cells (CTCs); and/or (h) amount and molecular features of circulating tumor DNA (ctDNA) in the blood.

In some embodiments, the subject exhibits a specific baseline pattern of genetic alterations (mutations, deletions, translocations, amplification) or gene/protein expression. In some embodiments, the subject exhibits a change from baseline in gene expression in peripheral blood (LSD1 regulated gene expression in PBMCs) and in tumor samples (such as SOX-2, Notch1/2, IGFBP2/5). In some embodiments, the subject exhibits a change from baseline in secreted proteins in blood selected from pro-gastrin-releasing peptide (pro-GRP) and chromogranin A (CgA). In some embodiments, the subject exhibits a change from baseline in localization and/or density of T cells in tumor tissues. In some embodiments, the subject exhibits a change from baseline in expression of programmed cell death protein 1 (PD-1) and/or programmed death-ligand 1 (PD-L1) in tumor tissues. In some embodiments, the subject exhibits a change from baseline in expression of lysine-specific histone demethylase 1A (LSD1) and/or an LSD1-associated molecular signature in tumor tissue. In some embodiments, the subject exhibits a change from baseline in protein markers in tumor tissues and/or in circulating tumor cells (CTCs). In some embodiments, the subject exhibits a change from baseline in amount and molecular features of circulating tumor DNA (ctDNA) in the blood. In some embodiments, the gene expression in peripheral blood and/or in tumor samples is the gene expression of sex determining region Y-box 2 (SOX2).

In some embodiments, the protein markers in tumor tissues is one or more of AC124319.1, ADAR, APOL6, ARID5B, ARL4A, ASCL1, AUTS2, B2M, BANK1, BATF2, BPGM, BST2, BTG1, C1R, C1S, CASP1, CASP3, CASP4, CASP7, CASP8, CHGA, CCL2, CCL5, CCL7, CD274 (PDL1), CD3, CD38, CD4, CD40, CD69, CD74, CD8, CD86, CDH2, CDKN1A, CFB, CFH, CIITA, CMKLR1, CMPK2, CMTR1, CSF2RB, CXCL10, CXCL11, CXCL9, DDX58, DDX60, DHX58, DLL1, DLL3, EIF2AK2, EIF4E3, EPSTI1, FAS, FCGR1A, FGFR1, FGFR13, FGL2, FPR1, GBP4, GBP6, GCH1, GPR18, GRP, GZMA, HELZ2, HERC6, HEY1, HES1, HIF1A, HLA-A, HLA-B, HLA-DMA, HLA-DQA1, HLA-DRB1, HLA-G, ICAM1, IDOL IFI27, IFI30, IF135, IF144, IFI44L, IFIH1, IFIT1, IFIT2, IFIT3, IFITM2, IFITM3, IFNAR2, IFL10RA, IGFBP2, IGFBP5, IL15, IL15RA, IL18BP, IL2RB, IL4R, IL6, IL7, IRF1, IRF2, IRF4, IRF5, IRF7, IRF8, IRF9, ISG15, ISG20, ISOC1, ITGAB, ITGB7, JAG1, JAK2, KAT2B, KLRK1, LAP3, LATS2, LCP2, LGALS3BP, LYSE, LYSMD2, MAGEC2, 1-MAR, MCSF, MDK, METTL7B, MT2A, MEHFD2, MVP, MX1, MX2, MYD88, M-CSF, NAMPT, NCOA3, NEUROD1, NFKB1, NFKBIA, NLRC5, NMI, NOD1, NOTCH1, NOTCH2, NUP93, OAS2, OAS3, OASL, OGFR, P2RY14, PARP12, PARP14, PDE4B, PD1, PELI1, PFKP, PIM1, PLA2G4A, PLSCR1, PML, PNP, PNPT1, POU2F3, PSMA2, PSMA3, PSMB10, PSMB2, PSMB8, PSMB9, PSME1, PSME2, PTGS2, PTPN1, PTPN2, PTPN6, RAPGEF6, RBCK1, RCOR2, REST, RIPK1, RIPK2, RNF31, RSAD2, RTP4, SAMD9L, SAMHD1, SECTM1, SELP, SERPINGLSGK1, SLAMF7, SLC25A28, SOCS1, SOCS3, SOD2, SOX2, SP110, SPPL2A, SRI, SSPN, ST3GAL5, ST8SIA4, STAT1, STAT2, STAT3, STAT4, TAP1, TAPBP, TDRD7, THBS1, TNFAIP2, TNFAIP3, TNFAIP6, TNFAIP10, TOR1B, TRAFD1, TRIM14, TRIM 21, TRIM25, TRIM 26, TXNIP, UBE2L6, UPP1, USP18, VAMP5, VAMP8, VEGF, VCAM1, WARS, WNT11, XAF1, XCL1, ZBP1, ZEB1, ZEB2, or ZNFX1.

In some embodiments, the subject has been previously treated with chemotherapy, such as platinum-based chemotherapy or a platinum-based chemotherapy doublet (e.g., cisplatin and etoposide). In some embodiments, the subject is currently being treated with chemotherapy, such as platinum-based chemotherapy or a platinum-based chemotherapy doublet (e.g., cisplatin and etoposide).

### V. The Response Evaluation Criteria In Solid Tumors (RECIST) Guidelines

As described herein, Response evaluation criteria in solid tumors (RECIST) is used to measure how a cancer patient responds to treatment. The following information is extracted/summarized from Eisenhauer, 2009, New Response Evaluation Criteria in Solid Tumors: Revised RECIST Guideline (Version 1.1) (Eur J Cancer. 2009 January; 45(2):228-47.) Please refer to the primary reference for further information.

At screening, tumor lesions/lymph nodes are categorized as measurable or non-measurable.

### Measurable Disease

Tumor Lesions. Must be accurately measured in at least one dimension (longest diameter in the plane of measurement is to be recorded) with a minimum size of:
10 mm by CT scan (CT scan slice thickness no greater than 5 mm)
10 mm caliper measurement by clinical exam (lesions which cannot be accurately measured with calipers should be recorded as non-measurable)
20 mm by chest X-ray

Malignant Lymph Nodes: To be considered pathologically enlarged and measurable, a lymph node must be ≥15 mm in short axis when assessed by CT scan (CT scan slice thickness recommended to be no greater than 5 mm). At baseline and in follow-up, only the short axis is measured and followed.

### Non-Measurable Disease

All other lesions, including small lesions (longest diameter <10 mm or pathological lymph nodes with ≥10 to <15 mm short axis) as well as truly non-measurable lesions. Lesions considered truly non-measurable include leptomeningeal disease, ascites, pleural or pericardial effusion, inflammatory breast disease, lymphangitic involvement of skin or lung, abdominal masses/abdominal organomegaly identified by physical exam that is not measurable by reproducible imaging techniques.

### Tumor Response Evaluation

Target lesions: When more than one measurable tumor lesion is present at baseline then all lesions up to a maximum of five lesions total (and a maximum of 2 lesions per organ) representative of all involved organs should be identified as target lesions and is recorded and measured at baseline. Target lesions should be selected on the basis of their size (lesions with the longest diameter), be representative of all involved organs, but in addition should be those that lend themselves to reproducible repeated measurements. Note that pathological nodes must meet the measurable criterion of a short axis of ≥15 mm by CT scan and only the short axis of these nodes contribute to the baseline sum. All other pathological nodes (those with short axis ≥10 mm but <15 mm) should be considered non-target lesions. Nodes that have a short axis <10 mm are considered non-pathological and should not be recorded or followed. At baseline, the sum of the target lesions (longest diameter of tumor lesions plus short axis of lymph nodes: overall maximum of 5) is to be recorded.

After baseline, a value should be provided on the eCRF for all identified target lesions for each assessment, even if very small. If extremely small and faint lesions cannot be accurately measured but are deemed to be present, a default value of 5 mm may be used. If lesions are too small to measure and indeed are believed to be absent, a default value of 0 mm may be used.

Non-target lesions: All non-measurable lesions (or sites of disease) plus any measurable lesions over and above those listed as target lesions are considered non-target lesions. Measurements are not required but these lesions should be noted at baseline and should be followed as “present,” “absent,” or “unequivocal progression.”

### Response Criteria

Target and non-target lesions are evaluated for response separately, and then the tumor burden as a whole is evaluated as the overall response.

Target Lesion Response: Target lesions are assessed as follows:
Complete Response (CR). Disappearance of all target lesions. Any pathological lymph nodes (whether target or non-target) must have reduction in short axis to <10 mm.
Partial Response (PR). At least a 30% decrease in the sum of diameters of target lesions, taking as reference the baseline sum diameters.
Progressive Disease (PD). At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).
Stable Disease (SD). Neither sufficient shrinkage to qualify for PR nor sufficient increase to qualify for PD, taking as reference the smallest sum of diameters while on study.

Non-target Lesion Response: Non-target lesions are assessed as follows:
Complete Response (CR). Disappearance of all non-target lesions and normalization of tumor marker level. All lymph nodes must be non-pathological in size (<10 mm short axis).
Non-CR/Non-PD. Persistence of one or more non-target lesion(s) and/or maintenance of tumor marker level above the normal limits.
Progressive Disease (PD). Unequivocal progression (see comments below) of existing non-target lesions. (Note: the appearance of one or more new lesions is also considered progression).

When the Subject Also Has Measurable Disease: In this setting, to achieve “unequivocal progression” on the basis of the non-target disease, there must be an overall level of substantial worsening in non-target disease such that, even in presence of SD or PR in target disease, the overall tumor burden has increased sufficiently to merit discontinuation of therapy. A modest “increase” in the size of one or more non-target lesions is usually not sufficient to quality for unequivocal progression status. The designation of overall progression solely on the basis of change in non-target disease in the face of SD or PR of target disease is therefore extremely rare.

When the Subject Has Only Non-measurable Disease: This circumstance arises in some Phase 3 trials when it is not a criterion of study entry to have measurable disease. The same general concepts apply here as noted above; however, in this instance there is no measurable disease assessment to factor into the interpretation of an increase in non-measurable disease burden. Because worsening in non-target disease cannot be easily quantified (by definition: if all lesions are truly non-measurable) a useful test that can be applied when assessing subjects for unequivocal progression is to consider if the increase in overall disease burden based on the change in non-measurable disease is comparable in magnitude to the increase that would be required to declare PD for measurable disease: i.e., an increase in tumor burden representing an additional 73% increase in “volume” (which is equivalent to a 20% increase diameter in a measurable lesion). Examples include an increase in a pleural effusion from “trace” to “large,” an increase in lymphangitic disease from localized to widespread, or may be described in protocols as “sufficient to require a change in therapy.” If “unequivocal progression” is seen, the subject should be considered to have had overall PD at that point. While it would be ideal to have objective criteria to apply to non-measurable disease, the very nature of that disease makes it impossible to do so: therefore, the increase must be substantial.

### Overall Response

Overall response should be assessed according to Table A for subjects with target lesions, and Table B for subjects with only non-target lesions.

TABLE A
Time Point Response: Subjects With Target (±Non-target) Disease

| Target Lesions | Non-target Lesion | New | Overall |
|---|---|---|---|
| Response | Response | Lesions | Response |
| CR | CR | No | CR |
| CR | Non-CR/non-PD | No | PR |
| CR | Not evaluated | No | PR |
| PR | Non-PD or not all | No | PR |
| | evaluated | | |
| SD | Non-PD or not all | No | SD |
| | evaluated | | |
| Not all | Non-PD | No | NE |
| evaluated | | | |
| PD | Any | Yes or No | PD |
| Any | PD | Yes or No | PD |
| Any | Any | Yes | PD |

CR = complete response, PR = partial response, SD = stable disease, PD = progressive disease, NE = inevaluable.

TABLE B
Time Point Response: Subjects With Non-target Disease Only

| Nontarget Lesions Response | New Lesions | Overall Response |
|---|---|---|
| CR | No | CR |
| Non-CR/non-PD | No | Non-CR/non-PD^{a)} |
| Not all evaluated | No | NE |
| Unequivocal PD | Yes or No | PD |
| Any | Yes | PD |

CR = complete response, PR = partial response, SD = stable disease, PD = progressive disease, NE = inevaluable.
^{a)}Non-CR/non-PD” is preferred over “stable disease” for non-target disease since SD is increasingly used as endpoint for assessment of efficacy in some trials so to assign this category when no lesions can be measured is not advised.

### Symptomatic Deterioration

Subjects with a global deterioration of health status requiring discontinuation of treatment without objective evidence of disease progression at that time should be reported as ‘symptomatic deterioration.’ Every effort should be made to document objective progression even after discontinuation of treatment. Symptomatic deterioration is not a descriptor of an objective response: it is a reason for stopping study therapy. The objective response status of such subjects is to be determined by evaluation of target and non-target disease.

### VI. Definitions

The following definitions are provided to facilitate understanding of certain terms used throughout this specification.

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

As used in the description of the invention and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The term “administering” as used herein includes prescribing for administration as well as actually administering and includes physically administering by the subject being treated or by another.

As used herein “subject,” “patient,” or “individual” refers to any subject, patient, or individual, and the terms are used interchangeably herein. In this regard, the terms “subject,” “patient,” and “individual” includes mammals, and, in particular humans. When used in conjunction with “in need thereof,” the term “subject,” “patient,” or “individual” intends any subject, patient, or individual having or at risk for a specified symptom or disorder.

As used herein, the phrase “therapeutically effective” or “effective” in context of a “dose” or “amount” means a dose or amount that provides the specific pharmacological effect for which the compound or compounds are being administered. It is emphasized that a therapeutically effective amount will not always be effective in achieving the intended effect in a given subject, even though such dose is deemed to be a therapeutically effective amount by those of skill in the art. For convenience only, exemplary dosages are provided herein. Those skilled in the art can adjust such amounts in accordance with the methods disclosed herein to treat a specific subject suffering from a specified symptom or disorder. The therapeutically effective amount may vary based on the route of administration and dosage form.

The terms “treatment,” “treating,” or any variation thereof includes reducing, ameliorating, or eliminating (i) one or more specified symptoms and/or (ii) one or more symptoms or effects of a specified disorder. The terms “prevention,” “preventing,” or any variation thereof includes reducing, ameliorating, or eliminating the risk of developing (i) one or more specified symptoms and/or (ii) one or more symptoms or effects of a specified disorder.

“Pharmaceutically acceptable salt” includes both acid and base addition salts. A pharmaceutically acceptable salt of any one of the substituted heterocyclic derivative compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, benzenesulfonic acid, salicylic acid, and the like. Exemplary salts thus include sulfates, pyrosulfates, bisulfates, sulfites, bisulfites, nitrates, phosphates, monohydrogen-phosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitro-benzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, e.g., Berge S. M. et al., Pharmaceutical Salts, J. Pharma. Sci. 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like.

### EXAMPLES

As used in the following examples, CC-90011 refers to besylate salt of 4-[2-(4-amino-piperidin-1-yl)-5-(3-fluoro-4-methoxy-phenyl)-1-methyl-6-oxo-1,6-dihydro-pyrimidin-4-yl]-2-fluoro-benzonitrile.

### Example 1: Oral Capsule of CC-90011

CC-90011 in capsules is available in appropriate strengths and capsule sizes, containing the active pharmaceutical ingredient in opaque, hard shell capsules.

### Example 2: A Study to Assess Safety and Efficacy of CC-90011 in Combination with Nivolumab in Subjects with Advanced Cancers

The primary objective of the study is to evaluate in each individual cohort the overall response rate in subjects with SCLC or sqNSCLC treated with CC-90011 in combination with nivolumab.

The secondary objectives are to evaluate in each individual cohort the following endpoints/outcomes in subjects with SCLC or sqNSCLC receiving CC-90011 in combination with nivolumab: evaluate the safety and tolerability, evaluate the duration of response, evaluate the investigator-assessed progression-free survival, and evaluate the overall survival.

Additional objectives of the study include:
Evaluate PD effects of CC-90011 on gene expression in peripheral blood and in tumor samples;
Evaluate PD effects of CC-90011 on levels of secreted proteins (such as Pro-GRP, CgA) in blood;
Evaluate localization and density of T cells in tumor biopsy tissues obtained at baseline and on-treatment;
Evaluate expression PD-1 and PD-L1, LSD1 and an LSD1-associated molecular signature in tumor biopsy tissues obtained at baseline and on-treatment;
Evaluate the expression of additional protein markers, such as SOX2, in tumor tissues (at baseline and on-treatment) as well as in circulating tumor cells (CTCs);
Evaluate the amount and molecular characteristics of circulating tumor DNA (ctDNA) and CTCs
Explore the relationship between PK, PD biomarkers and/or clinical outcomes of CC-90011 in combination with nivolumab;
Assess the preliminary efficacy of CC-90011 in combination with nivolumab based on Investigator-assessed immune-modified Response Evaluation Criteria in Solid Tumors (imRECIST).

Study endpoints are described in the below table.

TABLE C
Study Endpoints

| Endpoint | Name | Description | Timeframe |
|---|---|---|---|
| Primary | Overall response rate | The proportion of subjects in the treated | Every 6 weeks post C1D1 |
| | | population who had complete response | for the first 24 weeks and |
| | | (CR) or partial response (PR) as assessed | then every 8 weeks until |
| | | by Investigator review per RECIST v1.1 | disease progression, new |
| | | | anticancer therapy, death |
| | | | or withdrawal by subject. |
| Secondary | Safety and tolerability | Safety and tolerability are assessed from | Signature of informed |
| | | adverse events (using NCI CTCAE v5.0), | consent through 28 days |
| | | laboratory tests, vital signs, ECOG | after the last dose of CC- |
| | | performance status, physical exams, | 90011 and 100 days after |
| | | concomitant medications, and dose | the last dose of nivolumab. |
| | | modifications. | |
| | Duration of response | The time from the first occurrence of a | Every 6 weeks post C1D1 |
| | | documented objective response to the | for the first 24 weeks and |
| | | time of the first objectively documented | then every 8 weeks until |
| | | progression, as determined by | disease progression, new |
| | | Investigator review per RECIST v1.1, or | anticancer therapy, death |
| | | death from any cause, whichever comes | or withdrawal by subject. |
| | | first. | |
| | Progression-free | The time from first dose of study | Every 6 weeks post C1D1 |
| | survival | treatment to the date of the first | for the first 24 weeks and |
| | | objectively documented tumor | then every 8 weeks until |
| | | progression as assessed by Investigator | disease progression, new |
| | | review per RECIST v1.1 or death from | anticancer therapy, death |
| | | any cause, whichever occurs first. | or withdrawal by subject. |
| | Overall Survival | The time from first dose of IP to the date | From the first dose of IP to |
| | | of death due to any cause. | the date of death due to any |
| | | | cause. |
| | Pharmacodynamics/ | Evaluate and assess the following: | Baseline and at |
| | Biomarkers | PD effects of CC-90011 on gene | prespecified timepoints |
| | | expression in peripheral blood and in | until disease progression or |
| | | tumor samples. | until death, lost to follow- |
| | | PD effects of CC-90011 on levels of | up, withdrawal by subject |
| | | secreted proteins (such as Pro-GRP, CgA) | from study, or the end of |
| | | in blood | trial, whichever occurs |
| | | localization, density of T cells in tumor | first. |
| | | tissues (at baseline and on-treatment) | |
| | | expression of PD-1 and PD-L1 in tumor | |
| | | tissues (at baseline and on-treatment) | |
| | | expression of LSD1 and an LSD1- | |
| | | associated molecular signature in tumor | |
| | | tissue (at baseline and on-treatment) | |
| | | PD effects on expression of additional | |
| | | protein markers in tumor tissues as well as | |
| | | in circulating tumor cells (CTCs) (at | |
| | | baseline and on-treatment) | |
| | | amount and molecular features of | |
| | | circulating tumor DNA (ctDNA) in the | |
| | | blood of patients (at baseline and on- | |
| | | treatment) | |
| Exploratory | Pharmacokinetics | Assess the relationship between PK/PD | Cycle 1 and subsequent |
| | | biomarkers and clinical outcomes of CC- | Cycles at specified |
| | | 90011. | timepoints. |
| | Preliminary efficacy by | ORR, DoR, PFS, based on investigator | Every 6 weeks post C1D1 |
| | imRECIST | assessment of response using imRECIST | for the first 24 weeks and |
| | | | then every 8 weeks until |
| | | | disease progression, new |
| | | | disease therapy, death or |
| | | | withdrawal of consent |

Abbreviations: CgA = chromogranin A; ECOG = Eastern Cooperative Oncology Group; imRECIST = immune-modified Response Evaluation Criteria in Solid Tumors; LSD1 = lysine-specific histone demethylase 1A; NCI CTCAE = National Cancer Institute Common Terminology Criteria for Adverse Events; ORR = overall response rate; PD = pharmacodynamic; PK = pharmacokinetic; Pro-GRP = pro-gastrin-releasing peptide; RECIST = Response Evaluation Criteria in Solid Tumors; C1D1 = Cycle 1, Day 1; SOX2 = SRY (sex determining region Y)-box 2.

This study evaluates the ability of CC-90011 to increase response rates in 3 different lung cancer populations: PD-1 inhibitor naïve (Cohort A, SCLC) and a PD-1 inhibitor “experienced” (Cohort B, SCLC; and Cohort C, sqNSCLC) when given in combination with nivolumab. Cohort A tests the hypothesis that CC-90011 could enhance nivolumab responses in specific SCLC phenotypes. In this cohort, the proportion of enrolled subjects who respond to treatment is expected to increase by the action of CC-90011. Tumors with higher expression of an LSD1-associated molecular signature and low T infiltrating lymphocytes (TILs) may have the best response to the combination of ICI with CC-90011, based on CC-90011 hypothesized mechanism of action of increasing T cell infiltration into tumors. Cohorts B and C tests whether CC-90011 can mitigate acquired resistance to ICI in SCLC as well as sqNSCLC. For these cohorts, the trial is enrolling subjects who have an initial response or stable disease to ICI, but progress within the first 9 months after completion of the chemotherapy treatment.

Study Design: Approximately 135 subjects total globally are enrolled into one of the following cohorts in 2 stages:
Cohort A: SCLC in ICI naïve subjects
Cohort B: SCLC in ICI progressor subjects
Cohort C: sqNSCLC in ICI progressor subjects

All subjects receive CC-90011 60 mg per oral (PO) weekly on Days 1, 8, 15, and 22 of every 28-day cycle and nivolumab 480 mg IV every 4 weeks. CC-90011 should be administered first, before nivolumab administration, if possible. Treatment is continued for 3 cycles.

Subjects may begin screening up to 28 days before first dose of study treatment. Treatment must begin within 3 days of enrollment. Subjects are treated until death, progressive disease, unacceptable toxicity, withdrawal of consent from treatment, physician decision, or for up to 2 years. For subjects who progress with only brain metastasis, treatment with IP is stopped, but may be continued after completion of, and recovery from local radiation treatment per the Investigator's judgement.

In the event of discontinuation of the study treatments, subjects are followed in survival follow-up for up to 2 years until death, withdrawal of consent from the entire study, lost to follow-up, or end of study.

The End of Trial is defined as either the date of the last visit of the last subject to complete the post-treatment survival follow-up, or the date of receipt of the last data point from the last subject that is required for primary, secondary and/or exploratory analysis, as prespecified in the protocol, whichever is the later date.

Inclusion Criteria: Subjects must satisfy the following criteria to be enrolled in the study: (1) Subject is ≥18 years of age. (2) Subject with histological or cytological confirmation of extensive stage SCLC (ES SCLC) or Stage IIIb or IV sqNSCLC. (3) Subject has received one or two prior lines of therapies, defined as: (a) Cohort A (SCLC, ICI naïve): (i) At least 1 prior treatment including a platinum-based chemotherapy doublet; (ii) A minimum of 3 cycles of platinum-based chemotherapy in first line treatment, unless stopped at 2 cycles due to treatment-related toxicity; (b) Cohort B (SCLC, ICI progressors): (i) At least 1 prior first or second line treatment includes an ICI; (ii) If treatment includes an ICI as maintenance therapy, at least 1 cycle of ICI in maintenance should have been completed; (iii) At least 1 prior treatment including a platinum-based chemotherapy doublet; (iv) A minimum of 3 cycles of platinum-based chemotherapy, with or without ICI, in first line treatment, unless stopped at 2 cycles due to treatment-related toxicity; (v) Subject must have progressed during ICI therapy, defined as unequivocal progression on or within 3 months of the last dose of ICI therapy (if no subsequent therapy); (c) Cohort C (sqNSCLC, ICI progressors): (i) At least 1 prior first or second line treatment includes an ICI; (ii) If treatment includes an ICI as maintenance therapy, at least 1 cycle of ICI in maintenance should have been completed; (iii) At least 1 prior treatment including a platinum-based chemotherapy doublet; (iv) A minimum of 3 cycles of platinum-based chemotherapy, with or without an ICI, in first line treatment, unless stopped at 2 cycles due to treatment-related toxicity; (v) Subject must have progressed during ICI therapy, defined as unequivocal progression on or within 3 months of the last dose of ICI therapy (if no subsequent therapy). (4) Subject has progressed at the last line of therapy. (5) Subject has a measurable disease defined by RECIST v1.1. (5) Subject is able to swallow medication. (6) Subject must have: (a) Absolute neutrophil count (ANC)≥1.5×10⁹/L; (b) Hemoglobin (Hgb)≥9 g/dL (one-time blood transfusion is allowed); (c) Platelet (Plt) Count ≥150×10⁹/L; (d) White blood cells (WBC)≥2×10⁹ L; (e) Serum AST/serum glutamic oxaloacetic transaminase (SGOT) or ALT/serum glutamic pyruvic transaminase (SGPT)≤3×upper limit of normal (ULN) or ≤5×ULN if presence of liver metastases; (f) Total serum bilirubin ≤1.5×ULN (≤3×ULN, if Gilbert's syndrome or if indirect bilirubin concentrations are suggestive of extrahepatic source of the elevation); (g) Creatinine clearance (CrCl)≥60 mL/minute based on Cockcroft-Gault or modification of diet in renal disease (MDRD) or ≥60 mL/min/1.73 m².

Efficacy Assessment: Tumor assessments by CT scan or MRI of the chest, abdomen, and pelvis (pelvis if per local practice) should be performed at screening within 28 days prior to enrollment, and at every 6 weeks (±7 days) post Cycle 1 Day 1 for the first 24 weeks and every 8 weeks (±7 days) thereafter, until disease progression, start of new anticancer therapy, or withdrawal of consent by the subject from the entire study.

Brain imaging by CT scan with contrast or Mill should be performed at screening and as clinically indicated. For subjects who received PCI, the brain imaging performed prior to initiation of PCI, must occur within 56 days prior to enrollment. For subjects who did not receive PCI, brain imaging must occur within 28 days prior to enrollment.

To ensure sufficient ability to assess tumor response, the same imaging procedure should be used throughout the study for each subject, and these imaging studies must include all lesions assessed at baseline. Tumor assessments by CT scan or Mill should also be performed at any time if clinically indicated. Subjects who did not receive PCI treatment and with historical tumor scans evaluable per RECIST v1.1 performed within 28 days before enrollment need not repeat scans for the purposes of screening. Evaluation of response for therapeutic decisions should be performed using RECIST v1.1 guidelines by Investigator assessment, and imRECIST as an exploratory assessment.

### Interim Results

The interim results are: 38 patients have enrolled into the following cohorts in Stage 1: Cohort A (n=14, with 2 treated at 60 mg), Cohort B (n=14), and Cohort C (n=10). Stage 2 Cohort A is open for enrollment. Due to thrombocytopenia events observed, the Steering Committee has recommended to continue dosing at CC-90011 40 mg QW (originally 60 mg) in combination with nivolumab 480 mg Q4W.

Regarding responders, the following responses have been observed:
Cohort A: 2 confirmed partial response, 1 unconfirmed partial response;
Cohort B: 1 unconfirmed partial response; and
Cohort C: 1 unconfirmed partial response.

While certain embodiments have been illustrated and described, it should be understood that changes and modifications can be made therein in accordance with ordinary skill in the art without departing from the technology in its broader aspects as defined in the following claims.

1. A method of treating a subject having small cell lung cancer (SCLC) and/or squamous non-small cell lung cancer (sqNSCLC) comprising:
(a) administering to the subject an LSD-1 inhibitor; and
(b) concomitantly administering nivolumab;
wherein the LSD-1 inhibitor is a compound having the structure: 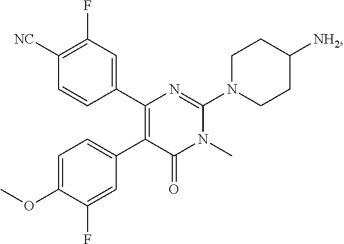 [SMILES: COc1ccc(-c2c(-c3ccc(C#N)c(F)c3)nc(N3CCC(N)CC3)n(C)c2=O)cc1F]
or a besylate salt thereof.

2. The method of claim 1, wherein the subject has any one of the following:
(a) a complete response (CR) as assessed by Response Evaluation Criteria In Solid Tumor (RECIST), Version 1.1;
(b) the disappearance of all target lesions; and/or
(c) the reduction of target and/or non-target pathological lymph nodes in short axis to less than about 10 mm.

3. The method of claim 1, wherein the subject has any one of the following:
(a) a partial response (PR) as assessed by Response Evaluation Criteria In Solid Tumor (RECIST), Version 1.1; and/or
(b) at least a 30% decrease in the sum of diameters of target lesions, taking as reference the baseline sum diameter.

4. The method of claim 1, wherein the subject has a duration of response as defined by a time from the first occurrence of a documented objective response to a time of a first objectively documented progression, as determined by Response Evaluation Criteria In Solid Tumor (RECIST), Version 1.1, or death from any cause, whichever comes first, wherein the duration of the response is:
(a) about 1, about 2, about 5, about 10, about 52, or greater weeks;
(b) at least about 1 week, at least about 2 weeks, at least about 3 weeks, at least about 4 weeks, at least about 5 weeks, at least about 6 weeks, at least about 12 weeks, at least about 18 weeks, at least about 24 weeks, at least about 30 weeks, at least about 36 weeks, at least about 42 weeks, at least about 48 weeks, or at least about 54 weeks; and/or
(c) about 1 week, about 2 weeks, about 3 weeks, about 4 weeks, about 5 weeks, about 6 weeks, about 12 weeks, about 18 weeks, about 24 weeks, about 30 weeks, about 36 weeks, about 42 weeks, about 48 weeks, or about 54 weeks.

5. The method of claim 1, wherein the subject has a progression-free survival as defined by from first dose of study treatment to the date of the first objectively documented tumor progression as determined by Response Evaluation Criteria In Solid Tumor (RECIST), Version 1.1, or death from any cause, whichever comes first, wherein the duration of the progression-free survival is:
(a) about 1, about 2, about 5, about 10, about 52, or greater weeks;
(b) at least about 1 week, at least about 2 weeks, at least about 3 weeks, at least about 4 weeks, at least about 5 weeks, at least about 6 weeks, at least about 12 weeks, at least about 18 weeks, at least about 24 weeks, at least about 30 weeks, at least about 36 weeks, at least about 42 weeks, at least about 48 weeks, or at least about 54 weeks; and/or
(c) about 1 week, about 2 weeks, about 3 weeks, about 4 weeks, about 5 weeks, about 6 weeks, about 12 weeks, about 18 weeks, about 24 weeks, about 30 weeks, about 36 weeks, about 42 weeks, about 48 weeks, or about 54 weeks.

6. The method of claim 1, further comprising any one of the following:
(a) the LSD-1 inhibitor is administered orally;
(b) the LSD-1 inhibitor is administered in the form of a tablet or a capsule;
(c) the LSD-1 inhibitor is administered once a week;
(d) the LSD-1 inhibitor is administered at a dose of about 20 mg, 40 mg, or 60 mg; and/or
(e) the LSD-1 inhibitor is administered at a dose of about 40 mg.

7. The method of claim 1, wherein:
(a) the LSD-1 inhibitor is administered at about 20 mg, about 40 mg, or about 60 mg orally once a week in a 28-day period; and/or
(b) the LSD-1 inhibitor is administered at about 40 mg orally once a week in a 28-day period; and/or
(c) the LSD-1 inhibitor is administered on Days 1, 8, 15, and 22 in a 28-day period; and/or
(d) the 28 day period is repeated for as long as the subject has a complete response (CR) or partial response (PR) as assessed by Response Evaluation Criteria In Solid Tumor (RECIST), Version 1.1.

8. The method of claim 1, wherein:
(a) the nivolumab is administered intravenously; and/or
(b) the nivolumab is administered in the form of an injection; and/or
(c) the nivolumab is administered once every two weeks or every 4 weeks; and/or
(d) the nivolumab is administered at a dose of at least about 240 mg or about 480 mg; and/or
(e) the nivolumab is administered at a dose of about 240 mg or about 480 mg.

9. The method of claim 1, wherein:
(a) the nivolumab is administered at about 480 mg intravenously once a week in a 28-day period; and/or
(b) the nivolumab is administered on Day 1 in a 28-day period; and/or
(c) the 28 day period is repeated for as long as the subject has a complete response (CR) or partial response (PR) as assessed by Response Evaluation Criteria In Solid Tumor (RECIST), Version 1.1.

10. The method of claim 1, wherein the subject exhibits the following change from baseline:
gene expression in peripheral blood (LSD1-regulated genes in PBMCs) and/or in tumor samples (SOX-2, Notch1, ASCL1, IGFBP2/5, REST, Hes1, Hey1, MDK, CgA, GRP).

11. The method of claim 10, wherein the gene expression in tumor samples is the gene expression of sex determining region Y-box 2 (SOX2).

12. The method of claim 17, wherein the protein markers in tumor tissues is one or more of AC124319.1, ADAR, APOL6, ARID5B, ARL4A, ASCL1, AUTS2, B2M, BANK1, BATF2, BPGM, BST2, BTG1, C1R, C1S, CASP1, CASP3, CASP4, CASP7, CASP8, CHGA, CCL2, CCL5, CCL7, CD274 (PDL1), CD3, CD38, CD4, CD40, CD69, CD74, CD8, CD86, CDH2, CDKN1A, CFB, CFH, CIITA, CMKLR1, CMPK2, CMTR1, CSF2RB, CXCL10, CXCL11, CXCL9, DDX58, DDX60, DHX58, DLL1, DLL3, EIF2AK2, EIF4E3, EPSTI1, FAS, FCGR1A, FGFR1, FGFR13, FGL2, FPR1, GBP4, GBP6, GCH1, GPR18, GRP, GZMA, HELZ2, HERC6, HEY1, HES1, HIF1A, HLA-A, HLA-B, HLA-DMA, HLA-DQA1, HLA-DRB1, HLA-G, ICAM1, IDO1, IFI27, IFI30, IFI35, IFI44, IFI44L, IFIH1, IFIT1, IFIT2, IFIT3, IFITM2, IFITM3, IFNAR2, IFL10RA, IGFBP2, IGFBP5, IL15, IL15RA, IL18BP, IL2RB, IL4R, IL6, IL7, IRF1, IRF2, IRF4, IRF5, IRF7, IRF8, IRF9, ISG15, ISG20, ISOC1, ITGAB, ITGB7, JAG1, JAK2, KAT2B, KLRK1, LAP3, LATS2, LCP2, LGALS3BP, LYSE, LYSMD2, MAGEC2, 1-MAR, MCSF, MDK, METTL7B, MT2A, MEHFD2, MVP, MX1, MX2, MYD88, M-CSF, NAMPT, NCOA3, NEUROD1, NFKB1, NFKBIA, NLRC5, NMI, NOD1, NOTCH1, NOTCH2, NUP93, OAS2, OAS3, OASL, OGFR, P2RY14, PARP12, PARP14, PDE4B, PD1, PELI1, PFKP, PIM1, PLA2G4A, PLSCR1, PML, PNP, PNPT1, POU2F3, PSMA2, PSMA3, PSMB10, PSMB2, PSMB8, PSMB9, PSME1, PSME2, PTGS2, PTPN1, PTPN2, PTPN6, RAPGEF6, RBCK1, RCOR2, REST, RIPK1, RIPK2, RNF31, RSAD2, RTP4, SAMD9L, SAMHD1, SECTM1, SELP, SERPINGLSGK1, SLAMF7, SLC25A28, SOCS1, SOCS3, SOD2, SOX2, SP110, SPPL2A, SRI, SSPN, ST3GAL5, ST8SIA4, STAT1, STAT2, STAT3, STAT4, TAP1, TAPBP, TDRD7, THBS1, TNFAIP2, TNFAIP3, TNFAIP6, TNFAIP10, TOR1B, TRAFD1, TRIM14, TRIM 21, TRIM25, TRIM 26, TXNIP, UBE2L6, UPP1, USP18, VAMP5, VAMP8, VEGF, VCAM1, WARS, WNT11, XAF1, XCL1, ZBP1, ZEB1, ZEB2, or ZNFX1.

13. The method of claim 1, wherein the subject exhibits the following change from baseline: secreted proteins in blood selected from pro-gastrin-releasing peptide (pro-GRP) and chromogranin A (CgA) and midkine.

14. The method of claim 1, wherein the subject exhibits the following change from baseline: localization and/or density of T cells, MDSCs and other immune cells in tumor tissues.

15. The method of claim 1, wherein the subject exhibits the following change from baseline: expression of programmed cell death protein 1 (PD-1) and/or programmed death-ligand 1 (PD-L1) in tumor tissues.

16. The method of claim 1, wherein the subject exhibits the following change from baseline: expression of lysine-specific histone demethylase 1A (LSD1) and/or an LSD1-associated molecular signature in tumor tissue.

17. The method of claim 1, wherein the subject exhibits the following change from baseline: protein markers in tumor tissues (such as CXCL9, MCSF, Notch1/2, chromagraninA) and/or in circulating tumor cells (CTCs).

18. The method of claim 1, wherein the subject exhibits the following change from baseline: amount and molecular features of circulating tumor DNA (ctDNA) in the blood.
